(19) United States
(12) Patent Application Publication
(10) Pub. No.: US 2024/0099665 A1
(43) Pub. Date: Mar. 28, 2024

(54) ELECTROCARDIOGRAM DATA PROCESSING SERVER, ELECTROCARDIOGRAM DATA PROCESSING METHOD OF EXTRACTING ANALYSIS REQUIRED SECTION WHILE SEGMENTING ELECTROCARDIOGRAM SIGNAL INTO SIGNAL SEGMENTS WITH VARIABLE WINDOW SIZES, AND COMPUTER PROGRAM

(71) Applicant: ATSENS CO., LTD., Gyeonggi-do (KR)

(72) Inventors: Kab Mun CHA, Gyeonggi-do (KR); Tae Youn KIM, Gyeonggi-do (KR); Byung Jin MOON, Gyeonggi-do (KR); Jong Ook JEONG, Gyeonggi-do (KR)

(73) Assignee: ATSENS CO., LTD., Gyeonggi-do (KR)

(21) Appl. No.: 18/473,432

(22) Filed: Sep. 25, 2023

(30) Foreign Application Priority Data
Sep. 26, 2022 (KR) 10-2022-0121967

(51) Int. Cl. A61B 5/00; A61B 5/352; G16H 50/20

(52) U.S. Cl. CPC A61B 5/7267; A61B 5/352; G16H 50/20

(57) ABSTRACT

The embodiments disclosed herein provide an electrocardiogram data processing server, an electrocardiogram data processing method, and a computer program. The embodiments disclosed herein further provide an electrocardiogram data processing server, an electrocardiogram data processing method, and a computer program, the electrocardiogram data processing server configured to determine whether analysis is required while segmenting an electrocardiogram signal into signal segments with variable window sizes, for instance, by changing a window size of a signal segment according to whether analysis of a previous signal segment is required.

### CROSS-REFERENCE TO RELATED APPLICATION

This application is based on and claims priority under 35 U.S.C. § 119 to Korean Patent Application No. 10-2022-0121967, filed on Sep. 26, 2022, in the Korean Intellectual Property Office, the disclosure of which is incorporated by reference herein in its entirety.

### BACKGROUND

### 1. Field

The disclosure relates to an electrocardiogram data processing server, a method of processing electrocardiogram data, and a computer program, and more particularly, to a method of extracting an analysis required section while segmenting an electrocardiogram signal into signal segments with variable window sizes, thereby reducing electrocardiogram data screening time.

### 2. Description of the Related Art

When the heart muscle contracts and relaxes, electrical depolarization and repolarization generate a potential difference, and the potential difference is detected by attaching a surface electrode to the skin, the result of detection being an electrocardiogram. The electrocardiogram has a magnitude of several tens of μV to several mV and a frequency band of less than 100 Hz.

Recently, an electrocardiogram signal is measured through a patch-type measuring device. The patch-type measuring device continuously records electrocardiogram signals for a long time (e.g., 7 to 14 days) while a patient leads a normal daily life. The capacity of the recorded electrocardiogram signal is very large, and analyzing all the recorded electrocardiogram signals takes much time.

In addition, the electrocardiogram signal includes sections in which analysis is not required due to noise at the time of measurement. Therefore, selecting an analysis required section for an electrocardiogram signal measured for a long time is needed.

The aforementioned background art is technical information kept by the inventor for derivation of the disclosure or obtained in the process of derivation of the disclosure and cannot necessarily be considered as known technology disclosed to the general public prior to filing the disclosure.

### SUMMARY

The embodiments disclosed herein provide an electrocardiogram data processing server, an electrocardiogram data processing method, and a computer program.

Embodiments disclosed herein provide an electrocardiogram data processing server, an electrocardiogram data processing method, and a computer program, the electrocardiogram data processing server configured to determine whether analysis is required while segmenting an electrocardiogram signal into signal segments with variable window sizes.

Embodiments disclosed herein may further provide an electrocardiogram data processing server, an electrocardiogram data processing method, and a computer program, the electrocardiogram data processing server configured to determine whether analysis is required by changing the window size of the signal segment according to whether analysis of the previous signal segment is required.

Embodiments disclosed herein may provide an electrocardiogram data processing server, an electrocardiogram data processing method, and a computer program, the electrocardiogram data processing server configured to change a reference value for determining whether analysis is required according to the variable window size.

Additional aspects will be set forth in part in the description which follows and, in part, will be apparent from the description, or may be learned by practice of the presented embodiments.

According to some embodiments, a method of extracting an analysis required section while segmenting an electrocardiogram signal into signal segments with variable window sizes includes receiving, by an electrocardiogram data processing server, an electrocardiogram signal, receiving an analysis requirement of a first signal segment of the electrocardiogram signal, when the analysis requirement is true, loading a second signal segment subsequent to the first signal segment on a signal segment having a first window size identical to a preset initial value and determining the analysis requirement of the second signal segment, and when the analysis requirement is false, loading the second signal segment subsequent to the first signal segment on the signal segment having a second window size greater than the window size of the first signal segment and determining the analysis requirement of the second signal segment, and based on the analysis requirement of the second signal segment, classifying the second signal segment to an analysis required section or a section not requiring analysis.

In at least one variant, the method of extracting the analysis required section further includes storing data about signal segments belonging to the analysis required section and data about signal segments belonging to the section not requiring analysis in a memory.

In at least one variant, in the determining of the analysis requirement, the analysis requirement of the second signal segment may be determined by selecting from a method used for extracting electrocardiogram characteristics and an algorithm used for classifying an electrocardiogram.

In another variant, when the second signal segment is detected to include a predetermined electrocardiogram characteristic through the method used for extracting the electrocardiogram characteristics or to include predetermined electrocardiogram classifying information through an algorithm used for classifying the electrocardiogram, the analysis requirement of the second signal segment may be determined to be true.

In another variant, the determining of the analysis requirement may further include extracting at least one peak greater than a peak reference value of the second signal segment and determining the analysis requirement by comparing a number of peaks with a preset first threshold value.

In another variant, in response to the window size of the second signal segment, the first threshold value may be increased or decreased.

In another variant, the determining of the analysis requirement may further include extracting at least one peak greater than or equal to a frequency reference value of the second signal segment and determining the analysis requirement by comparing the number of peaks with a preset second threshold value.

In another variant, in response to the window size of the second signal segment, the second threshold value may be increased or decreased.

In another variant, the method may further include further analyzing at least one signal segment classified as the analysis required section. In the analyzing, at least one signal segment classified as the analysis required section may be analyzed by using an analysis model generated by machine learning.

In another variant, the second window size may be greater than or equal to twice the size of the first window size. The first signal segment and the second signal segment may include overlapping sections. The method may further include transmitting the second signal segment of the analysis required section to an external device through a network.

In another variant, the method may further include transmitting the second signal segment of the analysis required section to an external device through a network to further analyze the second signal segment of the analysis required section in the external device. The method may further include storing the second signal segment of the analysis required section in a memory.

An electrocardiogram data processing server including a communication unit, a computer-readable memory, and a processor, wherein the processor is configured to determine an analysis requirement of a first signal segment of the electrocardiogram signal, when the analysis requirement is true, load a second signal segment subsequent to the first signal segment on a signal segment having a first window size identical to a preset initial value and determine the analysis requirement of the second signal segment, when the analysis requirement is false, load the second signal segment subsequent to the first signal segment on the signal segment having a second window size greater than the window size of the first signal segment and determine the analysis requirement of the second signal segment, and based on the analysis requirement of the second signal segment, classify the second signal segment as an analysis required section or a section not requiring analysis.

In another variant, the processor may further be configured to determine the analysis requirement of the first signal segment and the second signal segment by using a separate module. The separate module may be implemented with hardware.

A computer program may be stored in a medium to perform any one method according to the embodiments by using a computer.

Further, another method, another system, and a computer-readable recording medium recording a computer program for proceeding the method are further provided.

Other aspects, features, and advantages may become clear from the following drawings, the claims, and the detailed description of the disclosure.

### BRIEF DESCRIPTION OF THE DRAWINGS

The above and other aspects, features, and advantages of certain embodiments will be more apparent from the following description taken in conjunction with the accompanying drawings, in which:

FIG. 1 illustrates a data processing system including a server and a user terminal according to an embodiment;

FIG. 2 is a diagram for explaining detailed elements of an electrocardiogram data processing server according to embodiments;

FIG. 3 is a block diagram of an electrocardiogram data processor;

FIG. 4 is a view of a biosignal measuring system to obtain an electrocardiogram signal according to some embodiments;

FIG. 5 is a block diagram of the electrocardiogram signal measuring device.

FIG. 6 is a flowchart of a method of classifying an analysis required section of an electrocardiogram signal according to some embodiments;

FIG. 7 is a flowchart of a method of determining a signal segment having a variable window size, according to some embodiments;

FIG. 8 is an example of a window size of the signal segment according to the prior art;

FIG. 9 is an example of signal segments segmented according to embodiments; and

FIG. 10 is a view for explaining a processing process when the embodiments of the disclosure are used.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

The elements and actions of the description are explained in detail by referring to the figures.

As the disclosure allows for various changes and numerous embodiments, certain embodiments will be illustrated in the drawings and described in the detailed description. Effects and features of the disclosure, and methods for achieving them will be clarified with reference to embodiments described below in detail with reference to the drawings. However, the disclosure is not limited to the following embodiments and may be embodied in various forms.

Hereinafter, embodiments will be described in detail with reference to the accompanying drawings, wherein the same or corresponding elements are denoted by the same reference numerals throughout and a repeated description thereof is omitted.

In the specification, terms such as “training” and “learning” are not intended to refer to mental operations such as human educational activities, but refer to performing neural network computing or machine learning through computing according to procedures. “Training” and “learning” refer to the study of computer algorithms that automatically improve through experience.

In the specification, the terms “first” and “second” are not used in a limited sense and are used to distinguish one component from another component.

As used herein, the singular expressions “a,” “an,” and “the” are intended to include the plural forms as well, unless the context clearly indicates otherwise.

It will be further understood that the terms “comprises” and/or “comprising” used herein specify the presence of stated features or components, but do not preclude the presence or addition of one or more other features or components.

In the following embodiments, a module may include a unit implemented as hardware, software, or firmware, and may be used interchangeably with terms such as logic, logic block, component, or circuit, for example. A module may be an integrated element or a smallest unit or a portion thereof performing at least one function. For example, according to an embodiment, the module may be implemented in the form of an application-specific integrated circuit (ASCI).

Sizes of elements in the drawings may be exaggerated for convenience of explanation. For example, because sizes and thicknesses of elements in the drawings are arbitrarily illustrated for convenience of explanation, the disclosure is not limited thereto.

When a certain embodiment may be implemented differently, a specific process order may be performed differently from the described order. For example, two consecutively described processes may be performed substantially at the same time or performed in an order opposite to the described order.

Hereinafter, a biosignal is a signal related to the body, and includes biosignals such as body temperature, pulse rate, electrocardiogram, brain wave, respiration rate, number of steps, stress, hormones, amount of exercise, calories consumed, body fat, water content in the body, blood sugar level, blood pressure, etc.

Hereinafter, the biosignal may be an analog signal or a digital signal of the measured biosignal.

Hereinafter, a biosignal, which is a digital signal, refers to numerically processing a signal for the purpose of modifying or improving an information signal in a desired direction.

Hereinafter, an electrocardiogram signal refers to measuring and diagnosing an abnormal rhythm of the heart, wherein an abnormal rhythm generated by an abnormal heart control signal in the brain or an abnormal rhythm due to damage to a conductive tissue or a neural transmission pathway that carries electrical signals are measured.

An electrocardiogram signal may be a signal measured by a 1-channel measurement device.

A decision model or an analysis model may be learned by data labeled with an attachment location of the electrocardiogram signal. A decision model or an analysis model may be learned by data labeled with a subject of the electrocardiogram signal.

The labeled data may be data containing a label entered by a user or a machine.

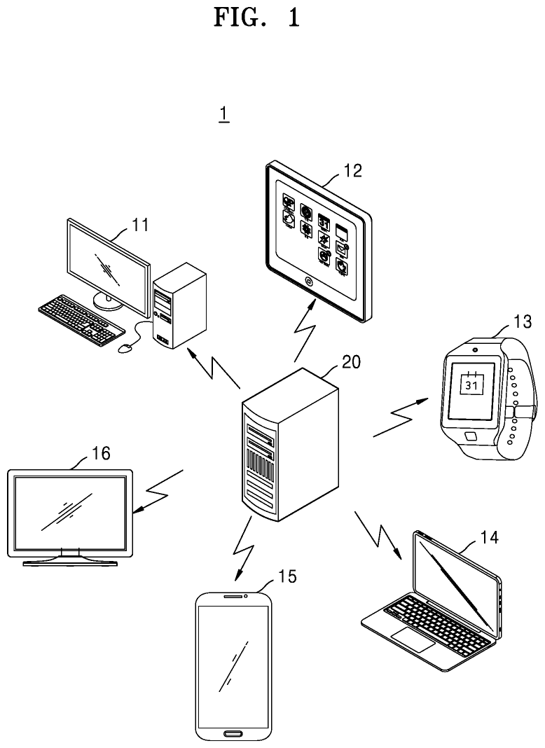

FIG. 1 illustrates an electrocardiogram data processing system including a server and a user terminal according to an embodiment of the present disclosure.

An electrocardiogram data processing system 1 of the present disclosure may include a server 20 and at least one user terminal 11 to 16. The server 20 may provide various online activities through a network. The server 20 may simultaneously provide online activities to at least one user terminal 11 to 16.

According to an embodiment, the server 20 may include a single server, a collection of servers, a cloud server, and the like, but is not limited to the above examples. The server 20 provides various medical online activities and may include a database storing data for network activities. In addition, the server 20 may include a payment server that generates and processes signal processing calculations or payment events. As described above, the server 20 may be an electrocardiogram data processing server.

According to an embodiment, a network refers to a connection established (or formed) using all communication methods and may refer to a communication network connected through all communication methods, the communication network transmitting and receiving data between terminals or between terminals and servers.

All communication methods may include communication through a predetermined communication standard, a predetermined frequency band, a predetermined protocol, or a predetermined channel. For example, Bluetooth, Bluetooth low energy (BLE), Wi-Fi, Zigbee, 3G, long-term evolution (LTE), communication methods through ultrasonic waves, etc. may be included, and short-distance communication, long-distance communication, wireless communication, and wired communication may all be included. Embodiments are not limited to the above examples.

According to an embodiment of the present disclosure, a short-distance communication method may refer to a communication method in which communication is possible only when a device (a terminal or server) performing communication is within a predetermined range, and for example, the device may include Bluetooth, near field communication (NFC), and the like. A long-distance communication method may refer to a communication method in which a communicating device is capable of communication regardless of distance. For example, the long-distance communication method may refer to a method in which two devices that communicate through a repeater such as an access point (AP) may communicate regardless of the distance between the two devices being greater than or equal to a predetermined distance and may include a communication method using a cellular network (3G, LTE) such as short message service (SMS) and phone. Embodiments are not limited to the above examples. Being provided with medical online activities using a network may mean that a communication between a server and a terminal may be performed through all communication methods.

At least one user terminal 11 to 16 in the entire specification may include various electronic devices such as personal digital assistants (PDA), a portable multimedia player (PNP), a navigation, an Mp3 player, a digital camera, a refrigerator, a washing machine, a cleaner, etc., in addition to a personal computer (PC) 11, a tablet 12, a cellular phone 13, a laptop 14, a smart phone 15, and a television (TV) 16. As described above, at least one user terminal 11 to 16 may be an electrocardiogram data processing server.

According to an embodiment, online activities may include data processing services, data analysis services, data distribution services, and data trading services, and are not limited to the above examples.

According to an embodiment, the server 20 may segment the electrocardiogram signal into a variable length and output whether or not the signal segment belonging to an analysis required section using a decision model. The length of a signal segment can be named window size. The server 20 may record data including signal segments belonging to the analysis required section. Here, the decision model is learned using machine learning, and can be learned using electrocardiogram signals measured in the past as input. The decision model can be learned using labeled ECG signals. The server 20 may classify the signal segments belonging to the analysis required section from a pre-stored electrocardiogram signal and generate analysis required data. In addition, server 20 may determine whether analysis of each signal segment of the electrocardiogram signal is required in the process of monitoring the electrocardiogram signal measured in real time and generate analysis required data. The server 20 may increase or decrease the length of first signal segment according to whether analysis of a previous signal segment is required using the decision model. A reference value for determining whether analysis of a signal segment is required may be determined using the decision. The reference value is a value determined as a result of learning labeled ECG signals. A signal segment classified into the analysis required section is equivalent to the signal segment for which analysis required is true. A signal segment classified into the section not requiring analysis may be equivalent to the signal segment for which analysis required is false.

The reference value may be changed according to the length of the signal segment. In another embodiment, the server 20 may determine whether analysis is required by comparing the number of peaks exceeding a peak reference value with a threshold value. The server 20 may classify the signal segments of which the analysis requirement is true 1 as the analysis required section. In addition, according to an embodiment, the electrocardiogram data processing system 1 may segment the electrocardiogram(ECG) signal into a variable length and output whether or not the signal segment belonging to an analysis required section using a decision model. The server 20 may record data including signal segments belonging to the analysis required section. The electrocardiogram data processing system 1 may classify the signal segments belonging to the analysis required section from a pre-stored electrocardiogram signal and generate analysis required data. In addition, the electrocardiogram data processing system 1 may determine whether analysis of each signal segment of the electrocardiogram signal is required in the process of monitoring the electrocardiogram signal measured in real time and generate analysis required data. The electrocardiogram data processing system 1 may increase or decrease a length of first signal segment according to whether analysis of a previous signal segment is required using the decision model. A reference value for determining whether analysis of a signal segment is required may be determined using the decision. The reference value is a value determined as a result of learning labeled ECG signals.

The reference value may be changed according to the length of the signal segment. In another embodiment, the electrocardiogram data processing system 1 may determine whether analysis is required by comparing the number of peaks exceeding a peak reference value with a threshold value. The electrocardiogram data processing system 1 may classify signal segments of which the analysis requirement is true as the analysis required section.

According to an embodiment, one of the at least one user terminal 11 to 16 may segment the electrocardiogram (ECG) signal into a variable length and output whether or not the signal segment belonging to an analysis required section using a decision model. The one of the at least one user terminal 11 to 16 may record data including signal segments belonging to the analysis required section. One of the at least one user terminal 11 to 16 may classify the signal segments belonging to the analysis required section from a pre-stored electrocardiogram signal and generate analysis required data. In addition, one of the at least one user terminal 11 to 16 may determine whether analysis of each signal segment of the electrocardiogram signal is required in the process of monitoring the electrocardiogram signal measured in real time and generate analysis required data. One of the at least one user terminal 11 to 16 may increase or decrease a length of first signal segment according to whether analysis of a previous signal segment is required using the decision model. A reference value for determining whether analysis of a signal segment is required may be determined using the decision. The reference value is a value determined as a result of learning labeled ECG signals.

The reference value may be changed according to the length of the signal segment. In another embodiment, One of the at least one user terminal 11 to 16 may determine whether analysis is required by comparing the number of peaks exceeding a peak reference value with a threshold value. One of the at least one user terminal 11 to 16 may classify signal segments of which the analysis requirement is true as the analysis required section.

The decision model for determining whether an analysis is required may be implemented to implement at least one of a method of extracting characteristics of an electrocardiogram signal or a method of classifying an electrocardiogram signal. Including a predetermined characteristic according to the method of extracting characteristics of the electrocardiogram signal or determining whether a predetermined analysis is required according to the method of classifying the electrocardiogram signal is performed in relation to every section of the electrocardiogram. Thus, the above steps may be performed according to an algorithm satisfying a predetermined standard. As for the decision model, an algorithm requiring short analyzing time and low power for analyzing may be determined.

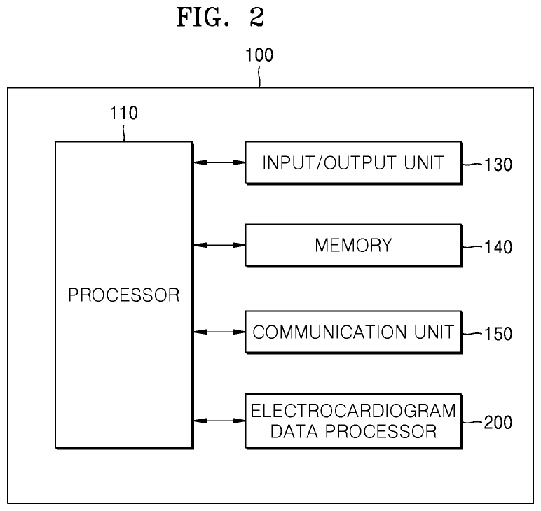

FIG. 2 is a diagram for explaining detailed elements of the electrocardiogram data processing server, according to some embodiments.

As shown in FIG. 2, an electrocardiogram data processing server 100 according to some embodiments may include a processor 110, an input/output unit 130, a memory unit 140, a communication unit 150, and an electrocardiogram data processor 200. However, not all elements shown in FIG. 2 are essential elements of the electrocardiogram data processing server 100. The electrocardiogram data processing server 100 may be implemented with more elements than those shown in FIG. 2, and the electrocardiogram data processing server 100 may be implemented with less elements than those shown in FIG. 2. The electrocardiogram data processing server 100 may be a user terminal, a server, an electrocardiogram data processing system, or a separate device.

According to an embodiment, the processor 110 usually controls the overall operation of the electrocardiogram data processing server 100. For example, the processor 110 may overall control elements included in the electrocardiogram data processing server 100 by executing a program stored in the electrocardiogram data processing server 100 or driving hardware provided therein.

According to an embodiment, the processor 110 may segment the electrocardiogram(ECG) signal into a variable length and output whether or not the signal segment belonging to an analysis required section using a decision model. The server 20 may record data including signal segments belonging to the analysis required section. The processor 110 may increase or decrease a length of first signal segment according to whether analysis of a previous signal segment is required. A reference value for determining whether analysis of a signal segment is required may be determined using the decision. The reference value is a value determined as a result of learning labeled ECG signals.

The reference value may be changed according to the length of the signal segment. In another embodiment, The processor 110 may determine whether analysis is required by comparing the number of peaks exceeding a peak reference value with a threshold value. The processor 110 may classify the signal segments of which the analysis requirement is true as the analysis required section.

The processor 110 is configured to overall control the electrocardiogram data processing server 100. Specifically, the processor 110 controls the overall operation of the electrocardiogram data processing server 100 by using various programs stored in the memory 140 of the electrocardiogram data processing server 100. For example, the processor 110 may include a central processing unit (CPU), random access memory (RAM), read-only memory (ROM), and system bus. Herein, the ROM stores an instruction set for system booting, and the CPU copies the stored operating system (O/S) of the electrocardiogram data processing server 100 on the RAM according to the instruction stored in the ROM and executes the O/S to boot the system. When the booting of the system is completed, the CPU may copy various stored applications to the RAM and perform various operations. In the above, the electrocardiogram data processing server 100 is described as including only one CPU but may be implemented as a plurality of CPUs (or DSP, SoC, etc.).

According to an embodiment, the processor 110 may be implemented as a digital signal processor (DSP) for processing digital signals, a microprocessor, or a time controller (TCON). However, embodiments are not limited thereto, and the processor 110 may include at least one of a CPU, a micro controller unit (MCU), a micro processing unit (MPU), a controller, an application processor (AP), or a communication processor (CP), and may be defined by the corresponding term. In addition, the processor 110 may be implemented as a system on chip (SoC) or a large scale integration (LSI) embedded with a processing algorithm, or may be implemented in the form of a field programmable gate array (FPGA).

According to an embodiment, the input/output unit 130 may display an interface generated by the memory 140 of the electrocardiogram data processing server 100. According to an embodiment, the input/output unit 130 may display a user interface of an input user input. The input/output unit 130 may output stored graphic data, visual data, auditory data, and vibration data by control of the memory 140.

The input/output unit 130 may be implemented in various forms of display panels. For example, the display panel may be implemented by various display technologies such as a liquid crystal display (LCD), organic light-emitting diodes (OLED), active-matrix organic light-emitting diodes (AM-OLED), liquid crystal on silicon (LCoS), or digital light processing (DLP). In addition, the input/output unit 130 may be coupled to at least one of the front surface area, side surface area, and rear surface area of the display panel in the form of a flexible display.

The input/output unit 130 may be implemented as a touch screen of a layer structure. The touch screen may not only have a display function, but also have functions of detecting a touch input location, a touch area, as well as a touch input pressure, and may not only perform a real-touch detecting function but also a proximity touch detecting function.

The input/output unit 130 may include a user interface for inputting various information in the electrocardiogram data processing server 100. In addition, the input/output unit 130 may be located at a remote site.

According to an embodiment, the memory 140 may store a program for processing and controlling the processor 110 and/or the electrocardiogram data processor 200 and may store data input and transmitted to the electrocardiogram data processing server 100 or data output from the electrocardiogram data processing server 100. According to an embodiment, the memory 140 may store information of a user account, health-related information, and/or heart related information. The memory 140 may include a database storing the above information.

According to an embodiment, the memory 140 may include at least one of a flash memory type storage medium, a hard disk type storage medium, a multimedia card micro type storage medium, card type memory (for example, secure digital (SD) memory, extreme digital (XD) memory, or the like), RAM, static random access memory (SRAM), ROM, electrically erasable programmable read-only memory (EEPROM), programmable read-only memory (PROM), magnetic memory, a magnetic disk, or an optical disk. In addition, according to an embodiment, the programs stored in the memory 140 may be classified into a plurality of modules according to the functions thereof. In addition, various types may be connected to the network.

According to an embodiment, the communication unit 150 may perform communication with an external device of the processor 110. For example, the communication unit 150 may communicate with an external device such as a payment server and an authentication server by controlling the processor 110. In addition, the communication unit 150 may obtain user information or user input through communication with an external interface.

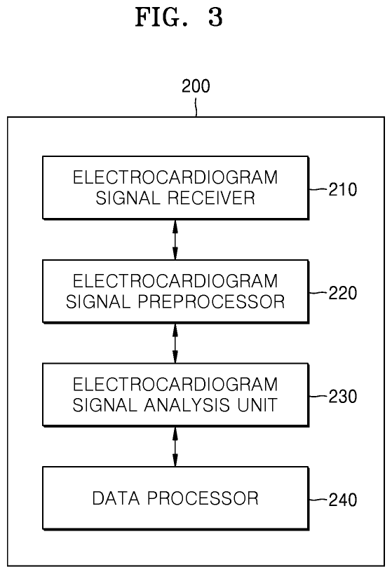

As shown in FIG. 3, the electrocardiogram data processor 200 may include an electrocardiogram signal receiver 210, an electrocardiogram signal preprocessor 220, an electrocardiogram signal analysis unit 230, and a data processor 240. All or part of the electrocardiogram signal receiver 210, the electrocardiogram signal preprocessor 220, the electrocardiogram signal analysis unit 230, and the data processor 240 may be implemented as at least one of software and hardware.

The electrocardiogram signal receiver 210 may receive an electrocardiogram signal. The electrocardiogram signal receiver 210 may receive an electrocardiogram signal from the database. The electrocardiogram signal receiver 210 may receive an electrocardiogram signal from the electrocardiogram measurement device. The electrocardiogram signal may be received from the communication unit 150, and the electrocardiogram signal may be used for certification and authentication to protect personal information.

The electrocardiogram signal preprocessor 220 may segment the electrocardiogram signal into signal segments to determine whether analysis of each signal segment is required and classify the signal segments of which the analysis requirement is true into the analysis required section. The electrocardiogram signal preprocessor 220 may execute the above process using the decision model. The decision model may be constructed to divide an electrocardiogram signal into signal segments using a fixed length division method, an overlap division method, a sliding window method, an adaptive method, etc. The electrocardiogram signal preprocessor 220 may transmit data related to the signal segment of the analysis required section to the electrocardiogram signal analysis unit 230.

The electrocardiogram signal preprocessor 220 may generate data that include within the analysis required section and data that does not include within a section not requiring analysis, in relation to the ECG signal, through the above process. The section not requiring analysis is a section that is not the analysis required section. The electrocardiogram signal preprocessor 220 may classify the electrocardiogram signal to the analysis required section and the section not requiring analysis to transmit only the electrocardiogram signal belonging to the analysis required section to the electrocardiogram signal analysis unit 230.

The electrocardiogram signal preprocessor 220 may segment the electrocardiogram signal into a plurality of signal segments and determine whether the analysis of each signal segment is required using the decision model. Determining whether analysis is required in the decision model may be performed considering the peaks included in the signal segment, the frequency value of the peak included in the signal segment, etc. However, embodiments are not limited thereto, and various methods for analyzing an electrocardiogram signal may be applied.

The electrocardiogram signal preprocessor 220 may determine whether analysis of the signal segment is required by extracting peaks greater than the peak reference value and determining whether the number of peaks is greater than or equal to a first threshold value. The peak reference value and/or the first threshold value may be determined by the decision model. The peak reference value may be determined in the process of learning electrocardiogram signals labeled with one of a heart disease suspect label, a noise label and a normal label. The peak reference value may be a reference value for determining signal peaks among an electrocardiogram signals. The first threshold value may be a reference value for determining whether analysis of a signal segment is necessary. The first threshold value may be determined based on the first unit threshold value set for a signal segment of a predetermined unit length. For example, a 20 second long signal segment may contain less than 25 signal peaks. For a 20 second long signal segment, the first threshold may be determined to be, for example, a value greater than 28 as a result of learning a plurality of signal segments. When the unit length is 20 seconds, the first unit threshold may be determined to be a value greater than 28. For example, the first threshold value may be determined by multiplying the ratio value of the window size of the signal segment by the first unit threshold value. Herein, the peak reference value may include a range of measurable data values in the electrocardiogram signal. The peak reference value may be determined by the measurable data values for each type of biosignal.

The electrocardiogram signal preprocessor 220 may convert second signal segment to a frequency dimension to extract first peak of which the frequency value is greater than or equal to a frequency reference value and may determine whether the number of the first peak is greater than or equal to a second threshold value to determine whether analysis of the second signal segment is required. The second threshold value may be determined based on the second unit threshold value set for a signal segment of a second unit length. For example, a second threshold value may be determined by multiplying the ratio value of the length of the signal segment by a second unit threshold value. The second unit threshold value may be determined by learning labeled electrocardiogram signals. Herein, a frequency reference value may include a range of measurable data values in the electrocardiogram signal. The frequency reference value may be determined by the measurable data values for each type of electrocardiogram signal.

Whether third signal segment is the analysis required section may be determined based on complexity values of RR interval lengths of the signal segment. The electrocardiogram signal preprocessor 220 may determine whether analysis of the third signal segment is required by comparing the complexity value of the RR interval length of the signal segment with a third threshold value. The third threshold value may be determined by learning labeled electrocardiogram signals.

Whether forth signal segment is the analysis required section may be determined based on the average value of RR interval lengths of the fifth signal segment.

The electrocardiogram signal preprocessor 220 may determine whether analysis of the forth signal segment is required by comparing the average value of the RR interval lengths of the forth signal segment with forth threshold value. Whether fifth signal segment is the analysis required section may be determined based on the standard deviation value of the RR interval lengths of the fifth signal segment.

The electrocardiogram signal preprocessor 220 may determine whether analysis of the fifth signal segment is required by comparing the standard deviation of the RR interval of the fifth signal segment with fifth threshold value.

The electrocardiogram signal preprocessor 220 may be implemented to change the length of the signal segment depending on whether a previous signal segment is the analysis required section. For example, when the previous signal segment is the analysis required section, a length of the signal segment can be set to be the same as a length of the previous signal segment.

If the previous signal segment is not the analysis required section, a length of the signal segment may be set to several times the length of the previous signal segment. The first to fifth thresholds may vary depending on the length (window size) of the signal segment. The previous signal segment and the signal segment may overlap in time.

In another embodiment, when the analysis requirement of the previous signal segment is false, the electrocardiogram signal preprocessor 220 may increase the length of the signal segment, and when the analysis requirement of the previous signal segment is true, the electrocardiogram signal preprocessor 220 may set the window size of the signal segment as a default value. The default value may be set by a user. The initial value may be set, for example, to 20 seconds.

The electrocardiogram signal analysis unit 230 may be implemented to analyze the signal segment classified as the analysis required section through the electrocardiogram signal preprocessor 220.

The electrocardiogram signal analysis unit 230 may generate analysis data regarding the signal segment by calculating a complexity value and/or a regularity value of signal segments classified as the analysis required section.

The electrocardiogram signal analysis unit 230 may be implemented to input the signal segment classified as the analysis required section to an analysis model to output analysis data regarding the signal segment using the analysis model.

Herein, the analysis model may be generated by using machine learning and data mining techniques. The model learned through machine learning can be made to infer the correlation between attribute values through a learning process and output analysis data based on an inferred algorithm. A method used to learn the analysis model may include supervised learning, self-learning, semi-supervised learning, reinforcement learning, advanced learning, etc. The artificial neural network is an algorithm inspired by the neural network of biology in machine learning and cognitive science, wherein artificial neurons forming a network by combining synapses change the binding force of the synapses through learning, resulting in a model having problem-solving abilities. The analysis model may be generated by learning through artificial neural networks.

The electrocardiogram signal analysis unit 230 may be implemented to analyze the signal segment at a frequency dimension by converting the signal segment classified as the analysis required section to the frequency dimension using the analysis model.

The electrocardiogram signal analysis unit 230 may analyze the signal segment classified as the analysis required section through various methods other than the above method.

The electrocardiogram signal analysis unit 230 may generate analysis data regarding the signal segment classified as the analysis required section.

The data processor 240 may convert and generate the signal segment classified as the analysis required section or analysis data of the signal segment in a predetermined processing method.

The data processor 240 may store the signal segment or analysis data of the signal segment in the memory or transmit the signal segment or analysis data of the signal segment to an external device. The data processor 240 may transmit the signal segment or analysis data of the signal segment to a predetermined device through network.

The above description is only an example, and various additional classifications or analysis processes may be further performed. The data processor 240 may analyze the electrocardiogram signal to detect the occurrence of risk situations. The analysis required section may be a section that includes a danger signal in the subject's biosignals. The timing at which the analysis required section is detected may be the point at which danger, disease, pain, or an unexpected event occurs in the subject's heart or breathing. The data processor 240 may generate an analysis report of the analysis required section. Herein, the analysis report may include a variety of data regarding the analysis required section. The analysis report may include markers of heart signals, abnormal heartbeats, poor supply of blood and oxygen to the heart, excessively thick heart muscle walls, and heart rate. Herein, the markers of the heart signals may be set to at least one of a normal beat N or a bundle branch block, a supraventricular ectopy beat S, and a ventricular ectopy beat VEB.

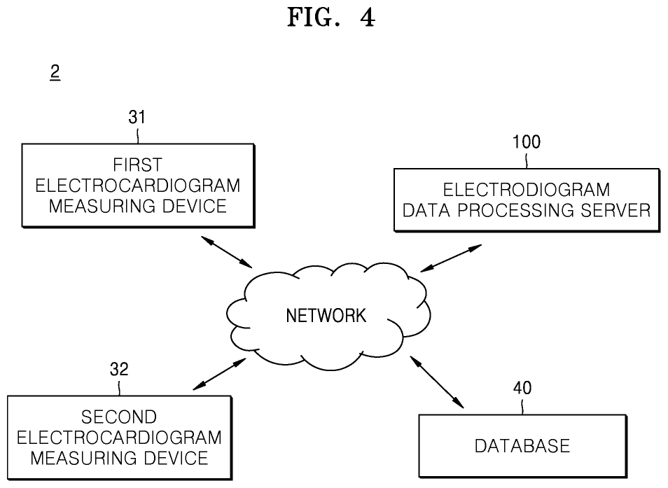

FIG. 4 is a view of a biosignal measuring system 2 to obtain an electrocardiogram signal according to some embodiments.

The biosignal measuring system 2 may include the electrocardiogram data processing server 100, a first electrocardiogram signal measuring device 31, and a second electrocardiogram signal measuring device 32. FIG. 4 illustrates the first electrocardiogram signal measuring device 31 and the second electrocardiogram signal measuring device, but additional electrocardiogram signal measuring devices may be included. The first electrocardiogram signal measuring device 31 and the second electrocardiogram signal measuring device 32 may be a patch-type measuring device or a halter-type measuring device.

The electrocardiogram data processing server 100 may receive a biosignal from a device such as a database 40. The electrocardiogram data processing server 100 may receive an electrocardiogram signal, a signal of the electrocardiogram signal processing, etc. from the first electrocardiogram signal measuring device 31, the second electrocardiogram signal measuring device 32, or the database 40. The electrocardiogram data processing server 100 may process the received electrocardiogram signal in response to the signal of the electrocardiogram signal processing.

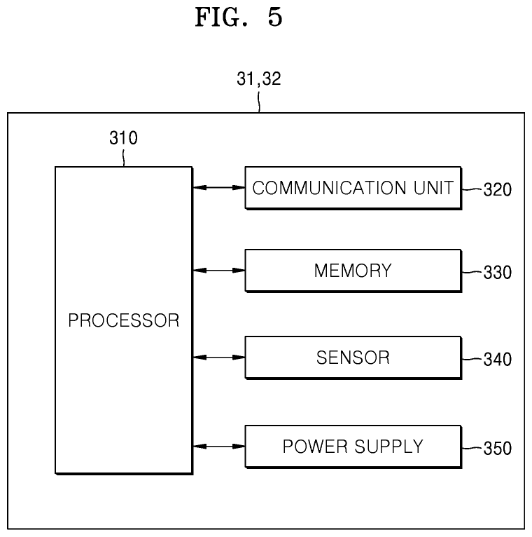

FIG. 5 is a block diagram of the electrocardiogram signal measuring device 31 and 32.

The electrocardiogram signal measuring device 31 and 32 may include a processor 310, a communication unit 320, a memory 330, a sensor 340, and a power supply 350. The electrocardiogram signal measuring device 31 or 32 may be mounted on a subject non-invasively or invasively to measure the electrocardiogram according to the heart rate of the subject. The electrocardiogram signal measuring device 31 or 32 may be a device that measures an electrocardiogram signal using two electrodes. The electrocardiogram signal measuring device 31 or 32 may be a wearable device worn on the wrist, but is not limited to this and refers to a device that measures electrocardiogram signals through one channel. The electrocardiogram signal measuring device 31 and 32 may be implemented as a form being attached to the skin or body of the subject but may not be limited thereto and implemented in various ways. Herein, the subject may be a person or animal, or a part of the body of a person or animal, such as the chest, but embodiments are not limited thereto and any object that can detect or measure the electrocardiogram may be a subject. In addition, the electrocardiogram is a graph of the potential changes on the body surface according to the mechanical activity of the heart rate, such as the contraction/expansion of the myocardium, and thus, “detecting the electrocardiogram” has the same meaning as “detecting the potential” generated on the body surface according to the heart rate of the subject.

The processor 310 may generally control elements such as the communication unit 320, the memory 330, the sensor 340, and the power supply 350. The processor 310 may store the electrocardiogram signal measured by the sensor 340 in the memory 330. Data such as electrocardiogram signals stored in the memory 330 may be transmitted to an external device. The processor 310 may receive a control signal from an external device to process data such as a biosignal according to the control signal. The processor 310 may process data such as the electrocardiogram signal in response to the control signal stored in the memory 330. The processor 310 may generate a biosignal processed or analyzed in response to the control signal.

The processor 310 may segment the electrocardiogram(ECG) signal into a variable length and output whether or not the signal segment belonging to an analysis required section using a decision model. The processor 310 may record data including signal segments belonging to the analysis required section. The processor 310 may determine whether the analysis of each signal segment of the electrocardiogram signal is required in the process of monitoring the electrocardiogram signal in real time and generate analysis required data.

The processor 310 may store the signal segments of which the analysis requirement is true in the memory.

The processor 310 may generate data requiring analysis including the analysis required section from the electrocardiogram signal. The processor 310 may classify the electrocardiogram signal to the analysis required section and the section not requiring analysis to transmit only the electrocardiogram signal belonging to the analysis required section to an external device.

The processor 220 may segment the electrocardiogram signal into a plurality of signal segments and determine whether the analysis of each signal segment is required using the decision model. Determining whether analysis is required in the decision model may be performed considering the peaks included in the signal segment, the frequency value of the peak included in the signal segment, etc. However, embodiments are not limited thereto, and various methods for analyzing an electrocardiogram signal may be applied.

The processor 310 may determine whether analysis of the signal segment is required by extracting peaks greater than the peak reference value and determining whether the number of peaks is greater than a first threshold value. The peak reference value and/or the first threshold value may be determined by the decision model. The peak reference value may be determined in the process of learning electrocardiogram signals labeled with one of a heart disease suspect label, a noise label and a normal label. The peak reference value may be a reference value for determining signal peaks among an electrocardiogram signals. The first threshold value may be a reference value for determining whether analysis of a signal segment is necessary. The first threshold value may be determined based on the first unit threshold value set for a signal segment of a predetermined unit length. For example, the first threshold value may be determined by multiplying the ratio value of the window size of the signal segment by the first unit threshold value. Herein, the peak reference value may include a range of measurable data values in the electrocardiogram signal. The peak reference value may be determined by the measurable data values for each type of biosignal.

The processor 310 may convert second signal segment to a frequency dimension to extract first peak of which the frequency value is greater than or equal to a frequency reference value and may determine whether the number of the first peak is greater than or equal to a second threshold value to determine whether analysis of the second signal segment is required. The second threshold value may be determined based on the second unit threshold value set for a signal segment of a second unit length. For example, a second threshold value may be determined by multiplying the ratio value of the length of the signal segment by a second unit threshold value. The second unit threshold value may be determined by learning labeled electrocardiogram signals. Herein, a frequency reference value may include a range of measurable data values in the electrocardiogram signal. The frequency reference value may be determined by the measurable data values for each type of electrocardiogram signal.

Whether third signal segment is the analysis required section may be determined based on complexity values of RR interval lengths of the signal segment. The processor 310 may determine whether analysis of the third signal segment is required by comparing the complexity value of the RR interval length of the signal segment with a third threshold value. The third threshold value may be determined by learning labeled electrocardiogram signals.

Whether forth signal segment is the analysis required section may be determined based on the average value of RR interval lengths of the fifth signal segment. The processor 310 may determine whether analysis of the forth signal segment is required by comparing the average value of the RR interval lengths of the forth signal segment with forth threshold value. Whether fifth signal segment is the analysis required section may be determined based on the standard deviation value of the RR interval lengths of the fifth signal segment. The processor 310 may determine whether analysis of the fifth signal segment is required by comparing the standard deviation of the RR interval of the fifth signal segment with fifth threshold value.

The processor 310 may be implemented to change the length of the signal segment depending on whether a previous signal segment is the analysis required section. For example, when the previous signal segment is the analysis required section, a length of the signal segment can be set to be the same as a length of the previous signal segment.

If the previous signal segment is not the analysis required section, a length of the signal segment may be set to several times the length of the previous signal segment. The first to fifth thresholds may vary depending on the length (window size) of the signal segment. The previous signal segment and the signal segment may overlap in time.

In another embodiment, when the analysis requirement of the previous signal segment is true, the processor 310 may increase the length of the signal segment, and when the analysis requirement of the previous signal segment is false, the processor 310 may set the window size of the signal segment as a default value. The default value may be set by a user. The initial value may be set, for example, to 20 seconds. The default value may be set by a user. The initial value may be set, for example, to 20 seconds.

In addition, the processor 310 may be implemented to analyze the signal segment classified as the analysis required section. The processor 310 may request for an additional analysis of the signal segment classified as the analysis required section to an external device. Here, the processor 310 may implement analysis processes as a separate specific hardware to reduce power consumption.

The processor 310 may be implemented as a digital signal processor (DSP), a microprocessor, or a time controller (TCON). However, embodiments are not limited thereto, and the processor 110 may include at least one of a CPU, a MCU, a MPU, a controller, an AP, or a CP, and an ARM processor, and may be defined by the corresponding term. In addition, the processor 310 may be implemented as a system on chip (SoC) or a large scale integration (LSI) embedded with a processing algorithm, or may be implemented in the form of a field programmable gate array (FPGA).

The communication unit 320 may communicate with other devices through a network.

The memory 330 may store the electrocardiogram signal sensed by the sensor 340. The memory 330 may store data about signal segments belonging to the analysis required section and data about signal segments belonging to the section not requiring analysis. The memory 330 may store data on reference values used to determine analysis required sections. The memory 330 may store a control signal for the electrocardiogram signal and store the electrocardiogram signal processed or analyzed in response to the control signal.

The sensor 340 detects changes in the physical and chemical phenomena that occur in humans or animals, and may sense values such as the body temperature, pulse, electrocardiogram, brain waves, respiratory rate, step count, stress, hormones, exercise amount, burned calories, body fat, body water content, blood sugar value, blood pressure, etc.

The power supply 350 may supply power to each element of the electrocardiogram signal measuring device 31 and 32.

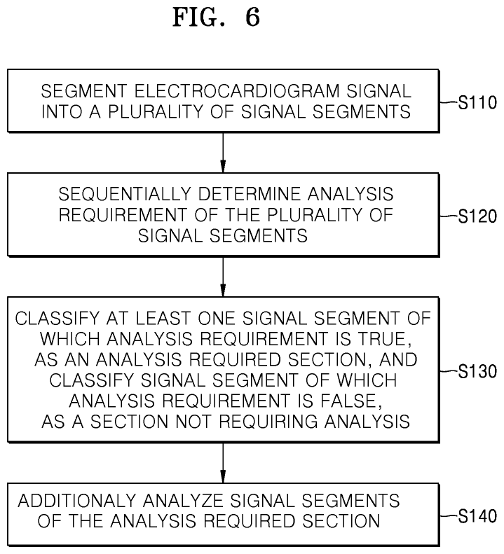

FIG. 6 is a flowchart of a method of classifying an analysis required section of an electrocardiogram signal according to some embodiments.

As shown in FIG. 6, in operation S110, the electrocardiogram data processing server 100 may be implemented to segment the electrocardiogram signal into signal segments according to a predetermined window size using the decision model.

In operation S120, the electrocardiogram data processing server 100 may execute the decision model to sequentially determine whether analysis is required for the signal segments.

In operation S130, the electrocardiogram data processing server 100 may classify one or more signal segments of which the analysis requirement is true as the analysis required section and may classify the signal segment of which the analysis requirement is false as the section not requiring analysis.

In operation S140, the electrocardiogram data processing server 100 may further analyze the signal segments of the analysis required section using the analysis model.

An electrocardiogram signal measured over 48 hours may include 8640 signal segments. That is, step S130 is executed 8640 times. Execution of steps 130 can be implemented to run for less time. Execution of steps 130 can be implemented to require less power. On the other hand, operation S140 is used for the conventional electrocardiogram analysis, wherein the analysis technique requiring analysis accuracy is used. Methods for analyzing electrocardiogram signals may include visual inspection, heart rate calculation methods, waveform interval measurement methods, morphological analysis methods, arrhythmia detection methods, ST segment analysis methods, machine learning, and AI utilization methods.

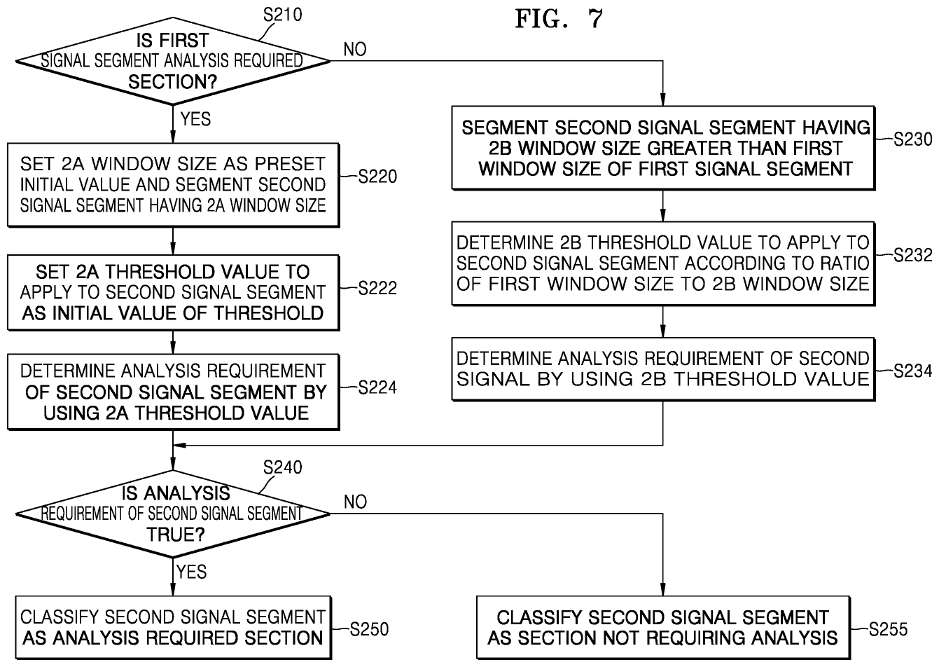

FIG. 7 is a flowchart of a method of determining a signal segment having a variable length, according to the embodiments.

In operation S210, the electrocardiogram data processing server 100 determines whether the first signal segment among the electrocardiogram signal is an analysis required section using the decision model. Here, the length of the first signal segment is named the first window size. It is assumed that the first threshold value is used when determining whether analysis of the first signal segment is required.

In operation S220, when the first signal segment is an analysis required section, the electrocardiogram data processing server 100 may set the 2A window size to the preset default value, and the second signal segment subsequent to the first signal segment may be segmented into the 2A window size.

In operation S222, the electrocardiogram data processing section 100 may set the 2A threshold value to be applied to the second signal segment as the default value of the first threshold value.

In operation S224, the electrocardiogram data processing server 100 may determine whether analysis of the second signal segment is required by using the 2A threshold value.

For example, the 2A threshold value may set value for the number of peaks of a signal segment that exceeds the peak reference value. In this case, whether analysis of the second signal segment is required may be determined through whether the number of peeks exceeding the peak reference value of the second signal segment exceeds the 2B threshold value.

Optionally, the 2B threshold value may be set value for a frequency reference value, an average R-R interval (average heart rate), or a deviation of the R-R interval (heart rate deviation). The 2B threshold value may be determined by learning labeled data.

In operation S230, if the first signal segment is not an analysis required section, the electrocardiogram data processing server 100 may determine a 2B window size of the second signal segment to be greater than the first window size of the first signal segment. For example, the 2B window size may be greater than twice the first window size.

In operation S232, the electrocardiogram data processing server 100 may determine a 2B threshold value to be applied to the second signal segment according to the ratio of the first window size to the 2B window size. For example, the 2B threshold value may be the first threshold value X (the 2B window size/the first window size). Alternatively, the 2B threshold value may be an initial value of the threshold value X (the initial value of the 2B window size/window size).

In operation S234, the electrocardiogram data processing server 100 may determine whether analysis of the second signal segment is required by using the 2B threshold value.

For example, the 2B threshold value may be set value for the number of peaks of the second signal segment that exceeds the peak reference value. In this case, whether analysis of the second signal segment is required may be determined through whether the number of peeks exceeding the peak reference value of the second signal segment exceeds the 2B threshold value.

Optionally, the 2B threshold value may be set value for a frequency reference value, an average R-R interval (average heart rate), or a deviation of the R-R interval (heart rate deviation).

In operation S240, the electrocardiogram data processing server 100 may determine whether the analysis requirement of the second signal segment is true.

If in the second signal segment peaks exceeding the peak reference value are included in exceed the 2A threshold value or the 2B threshold value, the second signal segment may be determined that the analysis of the second signal segment is required. Alternatively, if in the second signal segment peaks exceeding the frequency reference value are included in exceeding the 2A threshold value or the 2B threshold value, the second signal segment may be determined that the analysis of the second signal segment is required.

In operation S250, the electrocardiogram data processing server 100 may classify the second signal segment as an analysis required section when the analysis requirement of the second signal segment is true.

In operation S255, the electrocardiogram data processing server 100 may classify the second signal segment as a section not requiring analysis when the analysis requirement of the second signal segment is not true.

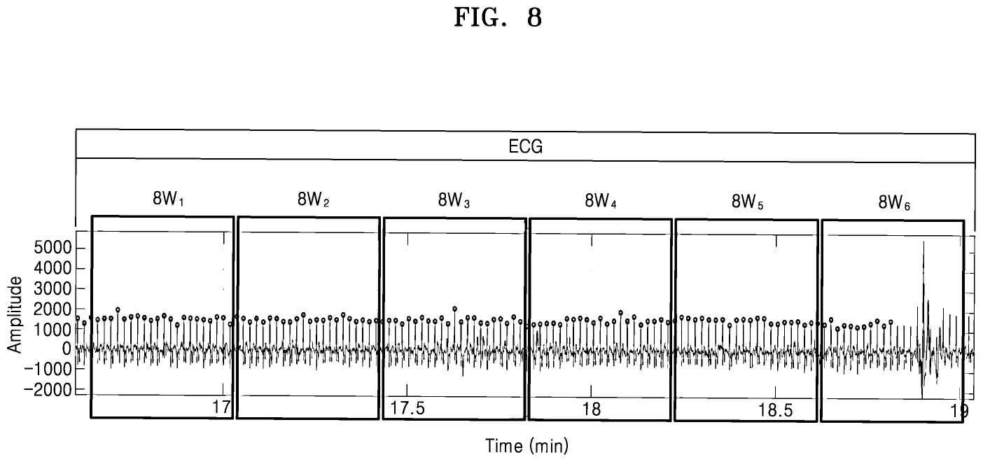

For convenience in explanation, the above descriptions determined whether the reference value was exceeded, but it is natural to use the reference value. For example, when determining by the reference range of the heart rate, whether analysis is required is determined within the predetermined range of upper and lower limits. When determining by the reference value of the heart rate deviation, whether analysis is required is determined within the predetermined range of the upper limit. If the heart rate deviation of a signal segment is less than or equal to the predetermined upper limit, the signal segment may be the section not requiring analysis. FIG. 8 is an example of a length of the signal segment according to the prior art.

According to the prior art, the electrocardiogram signal may be segmented into signal segments (8W1, 8W2, 8W3, 8W4, 8W5, and 8W6) having the same length.

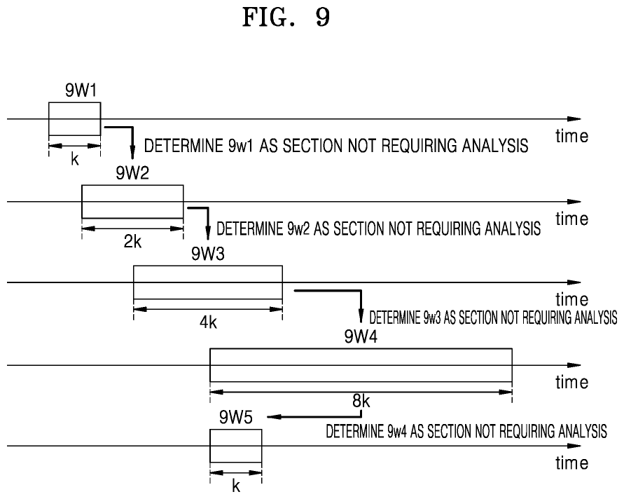

FIG. 9 is an example of signal segments segmented according to embodiments.

The electrocardiogram data processing server 100 may determine the analysis requirement of the signal segment 9W1 of the k length in the electrocardiogram signal. If the signal segment is determined to be a section not requiring analysis, the size of the subsequent signal segment 9W2 may be determined to be 2k, which is twice the length of the previous signal segment. The electrocardiogram data processing server 100 may determine whether analysis of the signal segment 9W2 is required by using a threshold value considering the length of 2k. For the signal segment of 2k, whether analysis is required may be determined by one operation.

If the signal segment 9W2 is determined to be a section not requiring analysis, the electrocardiogram data processing server 100 may segment the signal segment 9W3 by increasing the length of the subsequent signal segment to 4k. The electrocardiogram data processing server 100 may determine whether analysis of the signal segment 9W3 is required by using a threshold value considering the length of 4k. For the signal segment of 4k, whether analysis is required may be determined by one operation. Considering the above, the electrocardiogram data processing server 100 may classify the signal segment of 7k as the section not requiring analysis by three operations. Considering the prior art, the determining originally requiring seven operations may be performed in three operations. Accordingly, the calculation time to determine the analysis required section in the electrocardiogram signal may be shortened.

If the signal segment 9W3 is determined to be a section not requiring analysis, the electrocardiogram data processing server 100 may segment a signal segment 9W4 by increasing the length of the subsequent signal segment to 8k, which is twice the length of the previous signal segment. The electrocardiogram data processing server 100 may determine whether analysis of the signal segment 9W4 is required by using a threshold value considering the length of 8k.

If the signal segment 9W4 is determined to be an analysis required section, the electrocardiogram data processing server 100 may segment the signal segment 9W4 of the analysis required section into signal segments having lengths of k and determine again regarding the analysis required section. The electrocardiogram data processing server 100 may return to a starting point of the signal segment 9W4 and segment the signal segment 9W5.

The electrocardiogram data processing server 100 may segment the subsequent signal segments by setting the length of the subsequent signal segment to twice the length of the 9W5 when the analysis requirement 9W5 is false.

If the entire section of the signal segment 9W4 is determined to require analysis, the electrocardiogram data processing server 100 may determine whether the signal segment 9W4 requires through eight operations in the length of k. The eight operations are the greatest value, and if the signal segment 9W4 includes some analysis required sections, the analysis required section may be classified from the signal segment 9W4 with less than eight operations.

In FIG. 9, some of the signal segments are shown to overlap, embodiments are not limited thereto, and the signal segments may not overlap. Embodiments are not limited to the overlapping part of FIG. 9, and a very short signal segment may overlap.

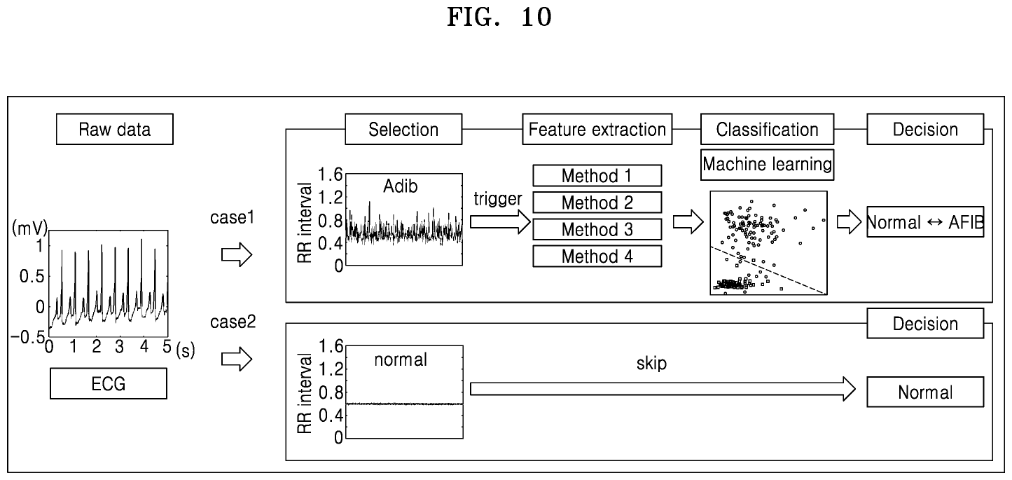

FIG. 10 is a view for explaining a processing process when the embodiments of the disclosure are used.

If the preprocessing process of the electrocardiogram signal according to some embodiments is used, among the signal segment of the electrocardiogram signal, the signal segment of the section not requiring analysis may be classified directly as a normal section.

The signal segment of the analysis required section may be classified as an abnormal section after going through additional analyzing processes (feature extraction and classification). The signal segment of the analysis required section may also be classified as the normal section.

Although the examples have been described with reference to the accompanying drawings, those of ordinary skill in the art will understand that various changes and modifications may be made therein. For example, the relevant results may be achieved even when the described technologies are performed in a different order than the described methods, and/or even when the described elements such as systems, structures, devices, and circuits are coupled or combined in a different form than the described methods or are replaced or substituted by other elements or equivalents.

Therefore, other implementations, other embodiments, and anything equal to the scope of claims fall within the scope of claims.

According to one of the embodiments above, provided are an electrocardiogram data processing server, an electrocardiogram data processing method, and a computer program.

In addition, an analysis requirement may be determined while segmenting an electrocardiogram signal into signal segments with variable window sizes.

In addition, the window size of the signal segment may be changed based on the analysis requirement of a previous signal segment to determine the analysis requirement.

In addition, a reference value for determining the analysis requirement according to the variable window size may be changed.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. A method of determining an analysis required section for signal segments with variable window sizes by an electrocardiogram data processing server including at least one processor, the method comprising:
receiving an electrocardiogram signal;
determining an analysis requirement of a first signal segment of the electrocardiogram signal having a first window size using a decision model;
determining a second window size of a second signal segment following the first signal segment depending on the analysis requirement of the first signal segment;
determining the analysis requirement of the second signal segment having a second window size using the decision model;
classifying each of the first and second signal segment as an analysis required section or a section not requiring analysis based on the analysis requirement of the first and second signal segment; and
storing, in a memory, data about signal segments belonging to the analysis required section and data about signal segments belonging to the section not requiring analysis.

2. The method of claim 1, wherein, the decision model is a model learned with labeled data using a machine learning.

3. The method of claim 2, further comprising implementing the decision model to determine each analysis requirement of each signal segment based on a number of peaks that is present within each signal segment and that exceeds a peak reference value.

4. The method of claim 3, further comprising determining the peak reference value by learning the labeled data.

5. The method of claim 1, further comprising setting the second window size to a value larger than the first window size upon determining that the analysis requirement of the first signal segment is false.

6. The method of claim 1, further comprising setting the second window size to a default value upon determining that the analysis requirement of the first signal segment is true.

7. The method of claim 1, further comprising implementing the decision model to determine the first or second signal segment as a section required analysis when a number of peaks, which is present within first or second signal segment and exceeds a peak reference value, exceeds a threshold.

8. The method of claim 7, further comprising further analyzing at least one signal segment classified as the analysis required section using an analysis model.

9. The method of claim 8, wherein, the analysis model is learned by machine learning from labeled data.

10. The method of claim 1, wherein the second window size is greater than or equal to twice the size of the first window size.

11. The method of claim 1, wherein the first and the second signal segment overlap in time.

12. The method of claim 5, further comprising re-determining the analysis requirement of the second signal segment after adjusting the second window size to a default value.

13. The method of claim 1, wherein the electrocardiogram signal is a signal measured by a 1-channel measurement device.

14. The method of claim 13, wherein the decision model is learned from data labeled with an attachment point of the electrocardiogram signal.

15. A server for determining an analysis required section for signal segments with variable window sizes, wherein the server comprises:
a communication unit that receives an electrocardiogram signal;
a memory unit that stores the electrocardiogram signal and generated data about signal segments belonging to an analysis required section and about signal segments belonging to a section not requiring analysis, and
a processor, wherein
the processor is configured to:
determine an analysis requirement of a first signal segment of the electrocardiogram signal having first window size using a decision model;
determine a second window size of a second signal segment following the first signal segment depending on whether the analysis requirement of the first signal segment;
determine the analysis requirement of the second signal segment having second window size using the decision model;
classify each of the first and second signal segment as the analysis required section or the section not requiring analysis based on the analysis requirement of the first and second signal segment; and
storing the data about the signal segments belonging to the analysis required section and the data about the signal segments belonging to the section not requiring analysis in the memory.

16. The server of claim 15, wherein the decision model is a model learned with labeled data using machine learning.

17. The server of claim 16, wherein the decision model is implemented to determine each analysis requirement of each signal segment based on a number of peaks that exists within each signal segment and that exceeds a peak reference value.

18. A non-transitory, computer-readable storage medium storing instruction that, when executed by a processor, causes the processor to perform operations comprising:
receiving an electrocardiogram signal;
determining an analysis requirement of a first signal segment of the electrocardiogram signal having first window size using a decision model;
determining a second window size of a second signal segment following the first signal segment depending on whether the analysis requirement of the first signal segment;
determining the analysis requirement of the second signal segment having second window size using the decision model;
classifying each of the first and second signal segment as an analysis required section or a section not requiring analysis based on the analysis requirement of the first and second signal segment; and
storing, in a memory, data about signal segments belonging to the analysis required section and data about signal segments belonging to the section not requiring analysis.
